(19) United States
(12) Patent Application Publication
(10) Pub. No.: US 2026/0021132 A1
(43) Pub. Date: Jan. 22, 2026

(54) PHARMACOLOGICAL THERAPY FOR MITOCHONDRIAL DNA DEPLETION DELETIONS SYNDROME INVOLVING MUTATIONS IN THE GUK1 GENE

(71) Applicant: The Trustees of Columbia University in the City of New York, New York, NY (US)

(72) Inventors: Agustin Hidalgo-Gutierrez, New York, NY (US); Michio Hirano, New York, NY (US); Jonathan Shintaku, New York, NY (US)

(21) Appl. No.: 19/339,836

(22) Filed: Sep. 25, 2025

Related U.S. Application Data
(63) Continuation of application No. PCT/US2024/021917, filed on Mar. 28, 2024.
(60) Provisional application No. 63/455,706, filed on Mar. 30, 2023.

(51) Int. Cl. A61K 31/708; A61K 31/519

(52) U.S. Cl. CPC A61K 31/708; A61K 31/519

(57) ABSTRACT

Compositions and methods relating to a pharmacological therapy for a human genetic disease, specifically mitochondrial DNA depletion-deletions syndromes, and more specifically, those related to mutations in the GUK1 gene. The pharmacological therapy involves the administration of deoxyguanosine (dG), a purine nucleoside phosphorylase (PNP) inhibitor, including but not limited to forodesine, or both.

### CROSS-REFERENCE TO RELATED APPLICATIONS

This application is a continuation of PCT International Application No. PCT/US2024/021917, filed Mar. 28, 2024, which claims benefit of U.S. Provisional Application No. 63/455,706, filed Mar. 30, 2023, the contents of each of which are hereby incorporated by reference.

### GOVERNMENT SUPPORT

This invention was made with government support under W81XWH2010807 awarded by the Department of Defense. The government has certain rights in the invention.

### BACKGROUND

Mitochondrial DNA (mtDNA) depletion-deletions syndromes (MDDS) encompass rare autosomal diseases characterized by reductions in mtDNA copy number and multiple deletions of mtDNA (Hirano, et al. 2001; Lopez-Gomez, et al. 2022). Depending on the patient's underlying primary nuclear gene mutation, MDDS affects skeletal muscle, brain, peripheral nerves, kidney, liver, and gastrointestinal tract. (Hirano, et al. 1994; Lopez-Gomez, et al. 2022; Oskoui, et al. 2006).

While some mutations causing MDDS directly impair mitochondrial DNA replication, another subset of genes causing MDDS are involved in nucleotide metabolism. MDDS disorders due to nucleotide metabolism and nucleotide pool imbalance can be amenable to pharmacological treatment.

### SUMMARY

Disclosed herein is a pharmacological therapy or treatment for MDDS patients with mutation(s) in GUK1, encoding guanylate kinase 1 (GUK1), an enzyme involved in purine metabolism. It is hypothesized that the impaired GUK1 is responsible for a novel form of MDDS through deoxynucleotide pool imbalance. Mutations in GUK1 and at least 12 additional nuclear genes cause nucleotide pool imbalances that lead to human MDDS that are disabling and lethal (Lopez-Gomez, et al. 2022).

Disclosed herein is a method of treating a disease or disorder characterized by unbalanced nucleotide pools or an MDDS related to GUK1, comprising administering to a subject in need thereof a therapeutically effective amount of a composition comprising one or more deoxynucleosides.

Diseases or disorders characterized by unbalanced nucleotide pools or MDDS that can be treated by the method are those characterized by mutations in GUK1. In some embodiments, the deoxynucleoside is deoxyguanosine (dG).

Disclosed herein is a method of treating a disease or disorder characterized by unbalanced nucleotide pools or an MDDS, comprising administering to a subject in need thereof a therapeutically effective amount of a composition comprising a purine nucleoside phosphorylase (PNP) inhibitor.

Diseases or disorders characterized by unbalanced nucleotide pools or MDDS that can be treated by the method are those characterized by mutations in GUK1.

In some embodiments, the purine nucleoside phosphorylase (PNP) inhibitor is a small molecule. In embodiments, the PNP inhibitor is a deoxyguanoside analog. In some embodiments, the purine nucleoside phosphorylase (PNP) inhibitor is forodesine.

Also disclosed herein is a method of treating a disease or disorder characterized by unbalanced nucleotide pools, or an MDDS comprising administering to a subject in need thereof a therapeutically effective amount of one or more compositions comprising one or more deoxynucleosides and a purine nucleoside phosphorylase (PNP) inhibitor.

Diseases or disorders characterized by unbalanced nucleotide pools or MDDS that can be treated by the method are those characterized by mutations in GUK1.

In some embodiments, the deoxynucleoside is deoxyguanosine (dG).

In some embodiments, the purine nucleoside phosphorylase (PNP) inhibitor is forodesine.

Dosages of the deoxynucleoside(s) can be between about 100 and about 1,000 mg/kg/day, between about 300 and about 800 mg/kg/day, or between about 250 and about 600 mg/kg/day.

Administration of the deoxynucleoside(s) and/or the PNP inhibitor can be once daily, twice daily, three times daily, four times daily, five times daily, up to six times daily, preferably at regular intervals.

Preferred methods of administration are oral, intrathecal, intravenous, and enteral. Administration of the deoxynucleoside(s) and/or the PNP inhibitor should begin as soon as the disorder characterized by unbalanced nucleotide pools, e.g., MDDS, is suspected and continue throughout the life of the patient.

In embodiments, the subject does not have a cancer. In embodiments, the cancer is T-cell lymphoma.

Disclosed is a composition comprising a therapeutically effective amount of a deoxyguanosine (dG) and a phosphorylase (PNP) inhibitor.

In embodiments, the composition is for treating a GUK1 deficiency in a subject. Also provided is use of the composition in the manufacture of a medicament for treating a GUK1 deficiency in a subject.

In embodiments, the phosphorylase (PNP) inhibitor is a small molecule PNP inhibitor.

In embodiments, the phosphorylase (PNP) inhibitor is forodesine.

Disclosed is a method comprising:
identifying a subject, or having a subject identified, as having a GUK1 mutation; and
administering to the subject (i) a therapeutically effective amount of a composition comprising deoxyguanosine (dG), (ii) a therapeutically effective amount of a composition comprising a phosphorylase (PNP) inhibitor, or (iii) a therapeutically effective amount of deoxyguanosine (dG) and a phosphorylase (PNP) inhibitor.

Disclosed is a method of increasing mtDNA in a subject with a GUK1 deficiency in a subject in need thereof comprising administering to the subject a therapeutically effective amount of (i) a composition comprising deoxyguanosine (dG), (ii) a composition comprising a phosphorylase (PNP) inhibitor, (iii) or one or two compositions comprising deoxyguanosine (dG) and a phosphorylase (PNP) inhibitor.

In embodiments, the methods comprise identifying the subject as having the mutation.

In embodiments, the subject has a mitochondrial DNA (mtDNA) depletion-deletions syndrome.

In embodiments, the mutation is a compound heterozygous mutation.

In embodiments, the mutation is p.Met1_fs; p.Gly11 Arg.

In embodiments, the subject has a GUK1 deficiency.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1: shows the reduced GUK1 enzyme activity of three sets of patient fibroblasts compared to controls (n=4). GUK1 enzyme activities were measured from proliferating fibroblasts as previously described (Hall, et al. 1986; Jong, et al. 1998) and normalized to total protein. *indicates p<0.05. P values determined by Mann-Whitney U test.

FIGS. 2A-2B: shows mtDNA content of (2A) proliferating GUK1 patient fibroblasts, and (2B) patient fibroblasts transfected with control or over-expression (o/e) plasmid. Levels of mtDNA were measured by quantitative Real-Time PCR (in triplicate) and expressed as mtDNA/nDNA (12S/RNase P) and levels are expressed as percent relative to control fibroblast mtDNA. * indicates p<0.05. P values determined by Mann-Whitney U test.

FIGS. 3A-3B shows mtDNA content of (3A) proliferating GUK 1 patient fibroblasts vs GUK 1 patient fibroblasts+dG 1 mM and 1 mM Forodesine together, and (3B) proliferating GUK1 patient fibroblasts vs GUK1 patient fibroblasts+dG 1 mM or 1 mM Forodesine separately. Levels of mtDNA were measured by quantitative Real-Time PCR (in triplicate) and expressed as mtDNA/nDNA (12S/RNase P) and levels are expressed as percent relative to control fibroblast mtDNA. * indicates p<0.05. P values determined by Mann-Whitney U test.

### DETAILED DESCRIPTION

The current disclosure is based upon the discovery of a novel pathway for mtDNA synthesis and causation of mitochondrial DNA depletion-deletions syndrome, characterized by mutations in GUK1. Moreover, the results disclosed herein indicate that the MDDS can be treated and/or prevented with the use of deoxyguanosine (dG), a purine nucleoside phosphorylase (PNP) inhibitor, or both.

A method of treating a GUK1 deficiency in a subject in need thereof comprising administering to the subject a therapeutically effective amount of a composition comprising deoxyguanosine (dG).

A method of treating a GUK1 deficiency in a subject in need thereof comprising administering to the subject a therapeutically effective amount of a composition comprising a phosphorylase (PNP) inhibitor.

A method of treating GUK1 deficiency in a subject in need thereof comprising administering to the subject a therapeutically effective amount of one or more compositions comprising deoxyguanosine (dG) and comprising phosphorylase (PNP) inhibitor.

In embodiments, no other dexoynucleoside besides dG are administered.

In embodiments, the phosphorylase (PNP) inhibitor is a small molecule PNP inhibitor.

In embodiments, the phosphorylase (PNP) inhibitor is forodesine.

In embodiments, the therapeutically effective amount of the composition comprising deoxyguanosine (dG) is between about 100 mg/kg/day and about 1000 mg/kg/day.

In embodiments, the therapeutically effective amount of the composition comprising deoxyguanosine (dG) is between about 200 mg/kg/day and about 800 mg/kg/day.

In embodiments, the therapeutically effective amount of the composition comprising deoxyguanosine (dG) is between about 250 mg/kg/day and about 400 mg/kg/day.

In embodiments, the composition or compositions are administered once daily, twice daily, three times daily, four times daily, five times daily or six times daily.

In embodiments the composition or compositions are orally, intrathecally, enterally, or intravenously.

In embodiments, the composition or compositions are administered orally and comprises deoxynucleoside and/or the PNP inhibitor mixed with cow's milk, human breast milk, infant formula, or water.

In embodiments, the one or more compositions are administered a plurality of times and the therapeutically effective amount of the one or more compositions administered to the subject is increased over time.

In embodiments, the subject is a human.

In embodiments, the subject does not have a cancer. In embodiments, the cancer is T-cell lymphoma.

A composition comprising a therapeutically effective amount of a deoxyguanosine (dG) and a phosphorylase (PNP) inhibitor.

In embodiments, the composition is for treating a GUK1 deficiency in a subject.

In embodiments, the phosphorylase (PNP) inhibitor is a small molecule PNP inhibitor.

In embodiments, the phosphorylase (PNP) inhibitor is forodesine.

A method comprising:
identifying a subject, or having a subject identified, as having a GUK1 mutation; and
administering to the subject (i) a therapeutically effective amount of a composition comprising deoxyguanosine (dG), (ii) a therapeutically effective amount of a composition comprising a phosphorylase (PNP) inhibitor, or (iii) a therapeutically effective amount of deoxyguanosine (dG) and a phosphorylase (PNP) inhibitor.

In embodiments, the method comprises identifying the subject as having the mutation. In embodiments, the method comprises identifying the subject as having the mutation by genetic analysis. In embodiments, the method comprises having the subject identified as having the mutation.

In embodiments, the subject has a mitochondrial DNA (mtDNA) depletion-deletions syndrome.

In embodiments, the mutation is a compound heterozygous mutation.

In embodiments, the mutation is p.Met1_fs; p.Gly11Arg.

In embodiments, the subject has a GUK1 deficiency.

A method of increasing mtDNA in a subject with a GUK1 deficiency in a subject in need thereof comprising administering to the subject a therapeutically effective amount of (i) a composition comprising deoxyguanosine (dG), (ii) a composition comprising a phosphorylase (PNP) inhibitor, (iii) or one or two compositions comprising deoxyguanosine (dG) and a phosphorylase (PNP) inhibitor.

In embodiments, the phosphorylase (PNP) inhibitor is a small molecule PNP inhibitor.

In embodiments, the phosphorylase (PNP) inhibitor is forodesine.

In embodiments, the therapeutically effective amount of the composition comprising deoxyguanosine (dG) is between about 100 mg/kg/day and about 1000 mg/kg/day.

In embodiments, the therapeutically effective amount of the composition comprising deoxyguanosine (dG) is between about 200 mg/kg/day and about 800 mg/kg/day.

In embodiments, the therapeutically effective amount of the composition comprising deoxyguanosine (dG) is between about 250 mg/kg/day and about 400 mg/kg/day.

In embodiments, the composition or compositions are administered once daily, twice daily, three times daily, four times daily, five times daily or six times daily.

In embodiments the composition or compositions are orally, intrathecally, enterally, or intravenously.

In embodiments, the composition or compositions are administered orally and comprises deoxynucleoside and/or the PNP inhibitor mixed with cow's milk, human breast milk, infant formula, or water.

In embodiments, the one or more compositions are administered a plurality of times and the therapeutically effective amount of the one or more compositions administered to the subject is increased over time.

In embodiments, the subject is a human. In embodiments, the subject does not have a cancer. In embodiments, the cancer is T-cell lymphoma.

In embodiments, mtDNA is increased in the subject sufficient to reduce one or more symptoms of an MDDS in the subject.

In embodiments, of the compositions, no other dexoynucleoside other than dG is present.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means mammals. Mammals include canines, felines, rodents, bovine, equines, porcines, ovines, and primates. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present disclosure, the “patient” is known or suspected of having a disease or disorder characterized by unbalanced nucleotide pools, mitochondrial disease, or mitochondrial DNA depletion-deletions syndrome, in some cases characterized by mutations in GUK1.

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder or slow its course of development.

The term “in need thereof” would be a subject known or suspected of having or being at risk of having a disease or disorder characterized by unbalanced nucleotide pools, mitochondrial disease, or mitochondrial DNA depletion-deletions syndrome, in some cases characterized by mutations in GUK1. GUK1 is known in the art, in and in a preferred embodiment the GUK1 is a human GUK1. Non-limiting examples of a human GUK1 gene are NCBI Gene ID: 2987; HGNC: 4693; and ENSG00000143774. The mutations impair the guanylate kinase function of the encoded guanylate kinase 1.

The term “agent” as used herein means a substance that produces or is capable of producing an effect and would include, but is not limited to, chemicals, pharmaceuticals, biologics, small organic molecules, antibodies, nucleic acids, peptides, and proteins.

The term “deoxynucleoside” as used herein means deoxythymidine or dT, deoxycytidine or dC, deoxyadenosine or dA, and deoxyguanosine or dG. The full-length name and common abbreviation for each will be used interchangeably. Such deoxynucleosides, unless otherwise stated, also include physiologically functional derivatives of the dexoynucleoside. In embodiments of the methods and compositions herein, the deoxynucleosides include physiologically functional derivatives of the dexoynucleoside. In embodiments of the methods and compositions herein, the deoxynucleosides include physiologically functional derivatives of the dexoynucleoside.

As used herein, the term “physiologically functional derivative” refers to a compound (e.g., a drug precursor) that is transformed in vivo to yield a deoxynucleoside. The transformation may occur by various mechanisms (e.g., by metabolic or chemical processes), such as, for example, through hydrolysis in blood. Prodrugs are such derivatives, and a discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

As used herein “an adverse effect” is an unwanted reaction caused by the administration of a drug. In most cases, the administration of the deoxynucleosides caused no adverse effects. The most expected adverse effect would be a minor gastrointestinal intolerance.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Mitochondrial DNA (mtDNA) depletion-deletions syndrome (MDDS) comprises several severe autosomal diseases characterized by a reduction in mtDNA copy number in affected tissues. Most of the MDDS causative nuclear genes encode proteins that belong to the mtDNA replication machinery or are involved in deoxyribonucleoside triphosphate (dNTP) metabolism.

One form of MDDS described herein is characterized by mutations in GUK1. GUK1 mutant patients have signs of involvement of skeletal muscle, heart, brain, intestine, and peripheral nerves. Symptoms of this MDDS can include failure to thrive; hepatomegaly; skeletal muscular symptoms such as ptosis, muscle atrophy, limb weakness and elevated creatine kinase; neurological and cognitive dysfunction symptoms such as autism spectrum disorder peripheral neuropathy and leukoencephalopathy; liver symptoms such as elevated AST/ALT and triglycerides; and immunodeficiency. Multiple mtDNA deletions and low mtDNA copy number are also seen. To date, four patients with GUK1 deficiency have been identified. However, it is anticipated that many more will be identified.

Additionally, experiments using both patient fibroblasts and a mouse model of GUK1 deficiency show the administration of deoxynucleosides and/or PNP inhibitor to be effective and safe for the treatment of the disease.

Thus, the present disclosure includes the administration of at least one deoxynucleoside and/or PNP inhibitor, or one or more compositions comprising at least one deoxynucleoside and/or PNP inhibitor to a patient in need thereof. In one embodiment, the patient is human. In one embodiment, the patient has an MDDS characterized by mutations in GUK1. In one embodiment, the deoxynucleoside is deoxyguanosine (dG). In one embodiment, the purine nucleoside phosphorylase (PNP) inhibitor is a small molecule. In one embodiment, the purine nucleoside phosphorylase (PNP) inhibitor is forodesine. In one embodiment, forodesine HCL is used.

Compositions comprising one of more deoxynucleosides and/or a PNP inhibitor can be pharmaceutical compositions. Such pharmaceutical compositions may comprise a therapeutically effective amount of the deoxynucleosides and/or a PNP inhibitor and a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans. “Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

Preferred methods of administration are oral, intrathecal and parental including intravenous. The compounds must be in the appropriate form for administration of choice.

Deoxynucleosides are easily dissolved in liquid, including commonly consumed liquids (such as water, formula or milk) whereas the free acid form does not readily dissolve in liquid.

Oral administration is a preferred method of administration. The deoxynucleosides and/or a PNP inhibitor can be added to any form of liquid a patient would consume. Examples include but are not limited to, mammalian milk, both cow's and human breast, plant or nut milks, infant formula, and water.

Additionally, pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

In order to overcome any issue of the deoxynucleosides and/or a PNP inhibitor crossing the blood/brain barrier, when delivery to the CNS is desired (e.g., treating a CNS manifestation of GUK1 deficiency), intrathecal administration is a further preferred form of administration (Galbiati, et al. 2006; Gotz, et al. 2008). Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well. In an embodiment of the methods, the method is for treating a CNS manifestation of GUK 1 deficiency. In an embodiment of the methods, the method is for treating one or more non-CNS manifestation(s) of GUK1 deficiency.

A further preferred form of administration is parenteral including intravenous administration. Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Additionally, since some patients may be receiving enteral nutrition by the time the deoxynucleoside treatment begins, the dNs (such as dG) and/or a PNP inhibitor can be administered through a gastronomy feeding tube or other enteral nutrition means.

Further methods of administration include mucosal, such as nasal, sublingual, vaginal, buccal, or rectal; or transdermal administration to a subject.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for transdermal administration may be provided as discrete patches intended to remain in intimate contact with the epidermis of the recipient over a prolonged period of time.

Selection of a therapeutically effective dose will be determined by the skilled artisan considering several factors, which will be known to one of ordinary skill in the art. Such factors include the particular form of the deoxynucleoside and/or PNP inhibitor, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

A dose of dG can range from about 50 mg/kg/day to about 500 mg/kg/day, or from about 50 mg/kg/day to about 700 mg/kg/day, or from about 50 mg/kg/day to about 900 mg/kg/day, or from about 100 mg/kg/day to about 1,000 mg/kg/day, or from about 100 mg/kg/day to about 800 mg/kg/day, or from about 100 mg/kg/day to about 600 mg/kg/day, or from about 100 mg/kg/day to about 400 mg/kg/day, or from about 200 mg/kg/day to about 800 mg/kg/day, or from about 200 mg/kg/day to about 600 mg/kg/day, or from about 200 mg/kg/day to about 400 mg/kg/day, or from about 250 mg/kg/day to about 800 mg/kg/day, or from about 250 mg/kg/day to about 600 mg/kg/day, or from about 250 mg/kg/day to about 400 mg/kg/day.

Administration of the deoxynucleosides and/or a PNP inhibitor can be once a day, twice a day, three times a day, four times a day, five times a day, up to six times a day, preferably at regular intervals. For example, when the deoxynucleosides are administered four times daily, doses would be at 8:00 AM, 12:00 PM, 4:00 PM, and 8:00 PM, with time determined to the time zone patient's location.

Doses can also be lower if being administered intravenously or intrathecally. Preferred dose ranges for such administration of dG are from about 50 mg/kg/day to about 500 mg/kg/day.

A subject can be monitored for improvement of their condition prior to increasing the dosage. A subject's response to the therapeutic administration of the deoxynucleosides and/or a PNP inhibitor can be monitored by observing a subject's muscle strength and control, and mobility as well as changes in height and weight. If one or more of these parameters increase after the administration, the treatment can be continued. If one or more of these parameters stays the same or decreases, the dosage of the deoxynucleosides and/or a PNP inhibitor can be increased.

It is known that these compounds individually are well tolerated. Any observed adverse effects were minor and were mostly diarrhea, abdominal bloating and other gastrointestinal manifestations. A subject can also be monitored for any adverse effects, such as gastrointestinal intolerance, e.g., diarrhea. If one or more adverse effects are observed after administration, then the dosage can be decreased. If no such adverse effects are observed, then the dosage can be increased. Additionally, once a dosage is decreased due to the observation of an adverse effect, and the adverse effect is no longer observed, the dosage can be increased.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Patient Fibroblasts

Two patients who are siblings with childhood-onset encephalomyopathy, share compound heterozygous mutations (p.Met1_fs; p.Gly11Arg) in GUK1, encoding guanylate kinase 1 (GUK1), an enzyme involved in purine metabolism. Mutations were initially identified by whole exome sequencing and confirmed by Sanger sequencing. In silico analyses with ConSurf and Polyphen2, along with structural mapping of these mutations, suggest these mutations have a “high functional impact.” These findings implicated a de novo nucleotide synthesis pathway in mtDNA maintenance.

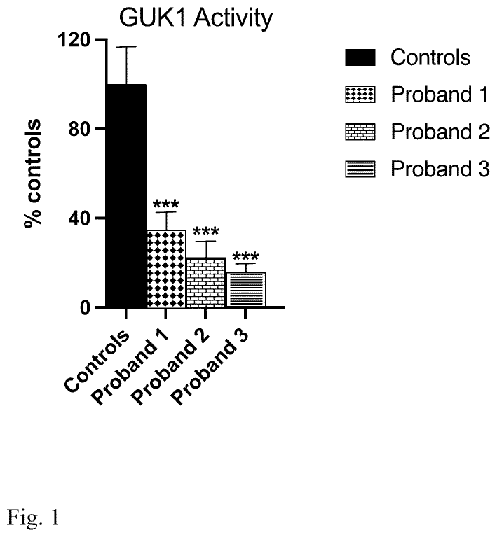

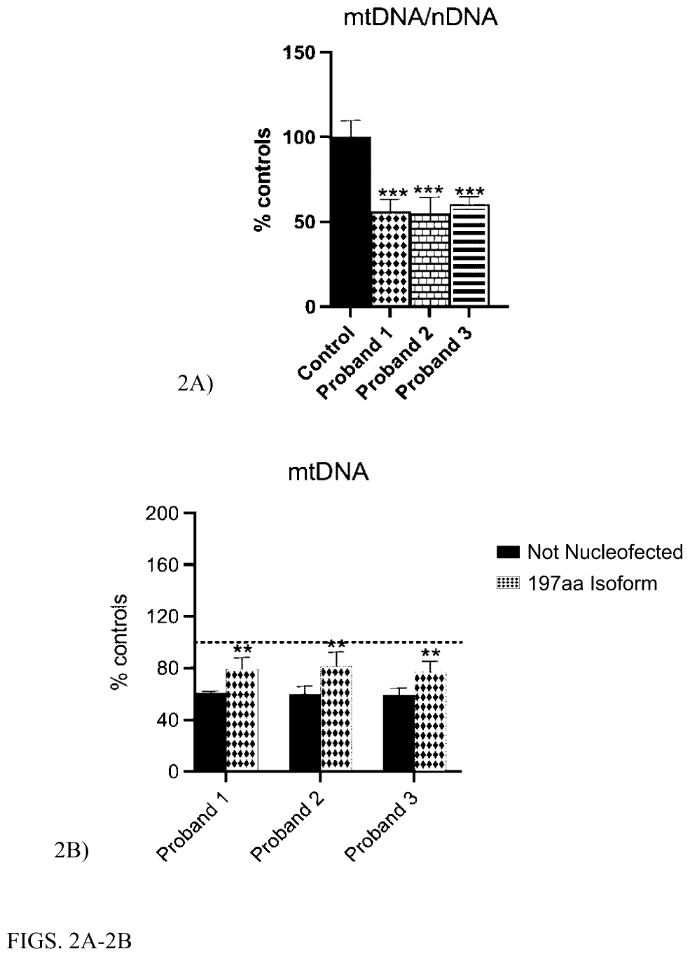

To characterize the consequences of the patients' mutations in vitro, primary skin fibroblasts from each patient were cultured. As expected, compared to fibroblasts obtained from healthy control subjects, GUK1-mutant patient fibroblasts have low guanylate kinase 1 activity (FIG. 1). In addition, the cells manifest abnormalities of mtDNA (FIG. 2) and nucleotide pool imbalance. Importantly, over-expression of normal GUK1 rescues mtDNA content in corresponding patient fibroblasts, further supporting the pathogenic nature of the identified GUK1 variants and the utility of this in vitro model (FIG. 2).

### Example 2—Mouse Model

A knock-in mice with a homologous p.Gly11Arg mutation (Ingenious Targeting Laboratory, Stony Brook, NY; Guk1KI/KI) has been generated. The targeted amino acid is highly conserved across species due to proximity to the ATP binding site, required for GUK1 activity. It is anticipated that this mutation disrupts GUK1 activity. A knock-out mouse has also been generated.

Tissues will be collected from mice to quantify Guk1 mRNA, protein, and enzyme activity. Examined tissues will include heart, skeletal muscle, liver, brain, small intestine, kidney, and spleen. If survival is affected, we will also assess post-mortem tissues for gross and histological changes. Skeletal muscle histology will include analysis of central nuclei, ragged-red fibers, and COX-SDH staining, all of which are consistent with a mitochondrial myopathy and were present in GUK1 patient biopsies. In addition, from these tissues, we will measure mtDNA copy number and integrity, mitochondrial and cytosolic dNTP pools, and OxPhos function. If survival is not affected, we will plan to examine tissues at 1, 3, 6, and 12 months of age, for progressive mtDNA depletion and deletions.

A common manifestation of mitochondrial diseases is myopathy manifesting as weakness, fatigue, and exercise intolerance. We will assess motor function as the Guk 1 mice age with grip and rotarod tests. A separate cohort of mice will undergo treadmill endurance tests. This could be useful both for measuring endurance and for possibly exacerbating disease symptoms, particularly if the mice do not have a clear phenotype. The stress of a treadmill endurance test would be analogous to patients with mtDNA depletion syndrome whose health can be significantly diminished by exercise, infection, or emotional stress. Mice will be monitored daily for signs of poor health that would call for euthanasia, including prominent lethargy, immobility, excessive running, stereotyped movements, and abnormal posture. We will also perform biweekly clinical evaluations including righting reflex, corneal reflex, salivation, and lacrimation of the eyes.

### Example 3—Pharmacological Therapy for GUK1 Deficiency

The multi-systemic disorder caused by GUK1-deficiency stems from the inability of cells to efficiently convert dGMP to dGDP, resulting in a deficit of dGTP and subsequent mtDNA depletion. We hypothesize that the mtDNA depletion caused by GUK1-deficiency can be overcome by administration of deoxyguanosine (dG), which will be converted to dGTP by the mitochondrial purine salvage pathway and residual GUK1 activity.

Initially, GUK1 mutant patient fibroblasts will be supplemented with dG. Based on published reports of deoxynucleoside supplementation for rescue of other models of mtDNA depletion, fibroblast culture media will be supplemented with dG at concentrations ranging from 1 μM to 400 μM (Camara, et al. 2014; Pontarin, et al. 2012; Taanman, et al. 2003; Bulst, et al. 2012). Throughout each experiment, fibroblasts will be actively proliferating to emphasize use of the de novo dNTP synthesis pathway. The primary outcome measure will be mtDNA copy number comparing untreated and dG-supplemented fibroblasts. To complement the mtDNA results, it will be determined whether the mitochondrial dGTP concentration increases with dG supplementation by measuring mitochondrial dNTP pools. As a functional measure of mitochondrial rescue in treated fibroblasts, we will also measure OxPhos activity. The anticipated result is rescue of both mitochondrial dGTP concentration and mtDNA copy.

Where dG supplementation does not rescue mitochondrial dGTP concentration or mtDNA sufficiently, to improve dG stability 0.5 μM of forodesine (Immucillin H), or other purine nucleoside phosphorylase (PNP) inhibitor, can be added. We have demonstrated in dGK-deficient fibroblasts that forodesine enhances the efficacy of purine supplementation (Camara, et al. 2014). Forodesine in mice can increase circulating dG (Bantia, et al. 2001). It may be beneficial alone or in combination with dG supplementation for treating GUK1-deficiency in vivo. Forodesine has been approved for clinical use in Japan for patients with relapsed or refractory T-cell lymphoma (Makita, et al. 2018). However, its use as an adjuvant therapy for GUK1-deficiency may be limited by toxicities, especially hematological effects, in humans. Therefore, assessments will be made to determine the lowest effective dose.

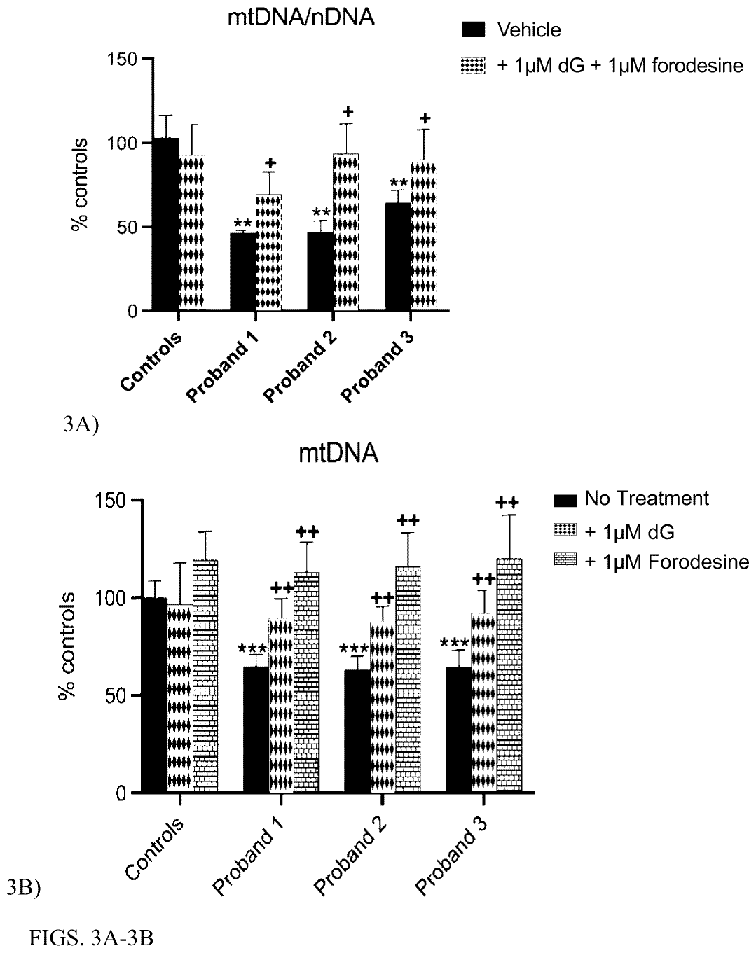

Initial experiments treating three different GUK1 patient proliferating fibroblasts with either dG at 1 μM or forodesine at 1 mM (FIG. 3B), or both (FIG. 3A) showed an increase in mtDNA as compared to untreated.

Treatment of the mice will follow a regimen of deoxynucleoside supplementation as follows. We will administer dG at concentrations of 300 and 600 mg/kg/day in sterile drinking water, with an estimated water intake of 5-6 mL/day. Initial treatments will continue 30 days beginning at postnatal day 4, followed by histological and molecular analyses, including tissue dNTP pool measurements, mtDNA quantitation, respiratory chain enzyme protein levels, and OxPhos activity. During the treatment period, in addition to monitoring survival and weight gain, we will also measure motor function, muscle strength, and endurance through use of rotarod, grip, and treadmill tests, and monitoring the function of tissues affected by GUK1-deficiency.

### REFERENCES

Bantia, et al. (2001) Purine nucleoside phosphorylase inhibitor BCX-1777 (Immucillin-H)—a novel potent and orally active immunosuppressive agent. Int Immunopharmacol. 1:1199-210
Bulst, et al. (2012) In vitro supplementation with deoxynucleoside monophosphates rescues mitochondrial DNA depletion. Mol Genet Metab. 107:95-103
Camara, et al. (2014) Administration of deoxyribonucleosides or inhibition of their catabolismas a pharmacological approach for mitochondrial DNA depletion syndrome. Hum Mol Genet. 23:2459-67
Galbiati, et al. (2006) New mutations in TK2 gene associated with mitochondrial DNA depletion. Pediatr. Neurol. 34:177-185
Gotz et al. (2008) Thymidine kinase 2 defects can cause multi-tissue mtDNA depletion syndrome. Brain 131:2841-2850
Hall, et al. (1986) Purification and properties of guanylate kinase from bovine retinas and rod outer segments. Eur J Biochem. 161:551-6. 2.
Hirano, et al. (2001) Defects of intergenomic communication: autosomal disorders that cause multiple deletions and depletion of mitochondrial DNA. Semin. Cell. Develop. Biol. 12:417-427
Hirano, et al. (1994) Mitochondrial neurogastrointestinal encephalomyopathy (MNGIE): clinical, biochemical, and genetic features of an autosomal recessive mitochondrial disorder. Neurology 44:721-7
Jong, et al. (1998) A simple and sensitive ribonucleotide reductase assay. J Biomed Sci. 5:62-8
Lopez-Gomez, et al. (2022) 232 ENMC International Workshop: recommendations for treatment of mitochondrial DNA maintenance disorders. 16-18 Jun. 2017, Heemskerk, The Netherlands. Neuromuscul. Disord. 32:609-20
Makita, et al. (2018) Forodesine in the treatment of relapsed/refractory peripheral T-cell lymphoma: an evidence-based review. Onco Targets Ther. 11:2287-93
Oskoui, et al. (2006) Clinical spectrum of mitochondrial DNA depletion due to mutations in the thymidine kinase 2 gene. Arch. Neurol. 63:1122-1126
Pontarin, et al. (2012) Mammalian ribonucleotide reductase subunit p53R2 58 is required for mitochondrial DNA replication and DNA repair in quiescent cells. Proc Natl Acad Sci USA. 109:13302-7
Taanman, et al. (2003) Mitochondrial DNA depletion can be prevented by dGMP and dAMP supplementation in a resting culture of deoxyguanosine kinase-deficient fibroblasts. Hum Mol Genet. 12:1839-45. 283

1. A method of treating a GUK1 deficiency in a subject in need thereof comprising administering to the subject (a) a therapeutically effective amount of a composition comprising deoxyguanosine (dG), or (b) a therapeutically effective amount of a composition comprising a phosphorylase (PNP) inhibitor, or (c) a therapeutically effective amount of one or more compositions comprising deoxyguanosine (dG) and comprising a phosphorylase (PNP) inhibitor.

2. The method of claim 1, wherein the phosphorylase (PNP) inhibitor is a small molecule PNP inhibitor.

3. The method of claim 2, wherein the phosphorylase (PNP) inhibitor is forodesine.

4. The method of claim 1, wherein the therapeutically effective amount of dG in the composition comprising dG is between about 100 mg/kg/day and about 1000 mg/kg/day.

5. The method of claim 1, wherein the therapeutically effective amount of dG in the composition comprising dG is between about 200 mg/kg/day and about 800 mg/kg/day.

6. The method of claim 1, wherein the therapeutically effective amount of dG in the composition comprising dG is between about 250 mg/kg/day and about 400 mg/kg/day.

7. The method of claim 1, wherein the composition or compositions are administered once daily, twice daily, three times daily, four times daily, five times daily or six times daily.

8. The method of claim 1, wherein the composition or compositions are administered orally, intrathecally, enterally, or intravenously.

9. The method of claim 8, wherein the composition or compositions are administered orally and comprises deoxynucleoside and/or the PNP inhibitor mixed with cow's milk, human breast milk, a nut or plant milk, infant formula, or water.

10. The method claim 1, wherein the one or more compositions are administered a plurality of times and the therapeutically effective amount of the one or more compositions administered to the subject is increased over time.

11. The method of claim 10, wherein the subject is a human.

12. The method of claim 11, wherein the subject does not have a cancer.

13. A composition comprising a therapeutically effective amount of a deoxyguanosine (dG) and a phosphorylase (PNP) inhibitor.

14. The composition of claim 13, for treating a GUK1 deficiency in a subject.

15. The composition of claim 14, wherein the phosphorylase (PNP) inhibitor is a small molecule PNP inhibitor.

16. The composition of claim 15, wherein the phosphorylase (PNP) inhibitor is forodesine.

17. A method comprising:
identifying a subject, or having a subject identified, as having a GUK1 mutation; and
administering to the subject (i) a therapeutically effective amount of a composition comprising deoxyguanosine (dG), (ii) a therapeutically effective amount of a composition comprising a phosphorylase (PNP) inhibitor, or (iii) a therapeutically effective amount of deoxyguanosine (dG) and a phosphorylase (PNP) inhibitor.

18. The method of claim 17, comprising identifying, by genetic analysis, the subject as having the mutation.

19. The method of claim 17, wherein the subject has a mitochondrial DNA (mtDNA) depletion-deletions syndrome.

20. The method of claim 19, wherein the mutation is a compound heterozygous mutation.
